(19) United States
(12) Patent Application Publication
(10) Pub. No.: US 2008/0004513 A1
(43) Pub. Date: Jan. 3, 2008

(54) VCSEL Tissue Spectrometer

(76) Inventors: Stephen D. Walker, Boulder, CO (US); Peter E. Nelson, Longmont, CO (US); R. Dale Zellers, Lafayette, CO (US); Charles W. Henry, Denver, CO (US); John E. Repine, Englewood, CO (US)

Correspondence Address: KOESTNER BERTANI LLP, 2192 Martin St., Suite 150, Irvine, CA, 92612, US

(21) Appl. No.: 11/428,217

(22) Filed: Jun. 30, 2006

(51) Int. Cl. A61B 5/00

(52) U.S. Cl. 600/310; 600/344; 600/316; 600/323

(57) ABSTRACT

In a tissue sensor, a plurality of emitters are configured to emit narrowband light into biological tissue at a plurality of selected wavelengths including one or more wavelengths indicative of a profile for one or more selected analytes and a reference wavelength at which absorbance for the selected analyte is negligible. The sensor further comprises a photo detector configured to detect light at wavelengths that emerge from the biological tissue.

### BACKGROUND OF THE INVENTION

Approximately thirty years ago, the medical device field experienced a massive technology shift. Sensors that could non-invasively monitor patient heart rate and oxygenation entered the marketplace and forever changed the standard by which all stakeholders in patient care viewed diagnostic devices. Sensors replaced the invasive process of using a syringe to extricate arterial blood which up to an hour later would generate only a single data point for a patient whose condition had often changed by the time the result was obtained. In today's world, what is desired is accurate data, generated immediately, continuously and painlessly. The preferences of physicians, nurses, hospital administrators, health insurers and especially patients are converging to drive the rapid and broad shift from the use of invasive testing to non-invasive and continuous monitoring. The shift is primarily sought by attributes of non-invasive monitoring including safety, less trauma to the patient, acquisition of continuous movie-like information rather than intermittent, single data points or snapshots. Non-invasive sensors reduce overall costs, are more convenient, and save time for hospital or clinical staff. Non-invasive sensors can also improve patient outcomes by supplying immediate data that can be used to care for unstable patients in a more timely fashion.

However, in the past thirty years, the ability to non-invasively monitor other parameters has not progressed beyond heart rate and oxygenation.

### SUMMARY

According to an embodiment of a tissue sensor, a plurality of emitters are configured to emit narrowband light into biological tissue at a plurality of selected wavelengths including one or more wavelengths indicative of a profile for one or more selected analytes and a reference wavelength at which absorbance for the selected analyte is negligible. The sensor further comprises a photo detector configured to detect light at wavelengths that emerge from the biological tissue.

### BRIEF DESCRIPTION OF THE DRAWINGS

Embodiments of the invention relating to both structure and method of operation may best be understood by referring to the following description and accompanying drawings:

FIG. 1 is a schematic block diagram that depicts an embodiment of a tissue sensor configured to monitor multiple physiological parameters;

FIGS. 2A and 2B are an overhead pictorial view and a cross-sectional cut-away view illustrating an embodiment of a tissue sensor in a configuration as part of a critical care monitor;

FIGS. 3A through 3F are flow charts showing several embodiments of aspects of embodiments of a method for noninvasively measuring the concentration of at least one analyte;

FIG. 4 is a pictorial view illustrating an embodiment of a possible configuration of a wearable Near Infrared Spectrometry (NIRS) sensor; and

FIGS. 5A and 5B are schematic flow charts depicting embodiments of methods for measuring analyte concentration.

### DETAILED DESCRIPTION

Near Infrared Spectrometry (NIRS) technology can be used in a tissue sensor and associated sensing method for noninvasive monitoring devices. The NIRS technology replaces conventional pulse oximeters with system and technique that can dramatically expand the number of analytes measured and tracked on a real-time basis.

NIRS technology further enables expanded analyte sensing and analysis in a compact, cost-competitive format.

Other existing optical technologies do not expand beyond the parameters of pulse oximeters due to the presence of water in tissue. Water absorbs a great deal of near infrared light and has thus thwarted attempts to noninvasively measure additional analytes. An illustrative tissue sensor greatly reduces or eliminates water interference, thereby creating a “window into the body”. Optics and an analytical algorithm combine with NIRS technology to generate readings across the near infrared spectrum and neutralize the masking effects of water.

NIRS technology is used to supply sensing of one or more physiological parameters in a Critical Care Monitor (CCM). The illustrative CCM is a NIRS-based device capable of non-invasively and continuously measuring a battery of factors useful for monitoring clinically relevant changes that occur rapidly and unpredictably in patients with acute inflammation disorders.

NIRS sensing is used in combination with an analytical technique that compares signals evoked by emitters at different wavelengths rather than simply measuring at a single wavelength. The technique analyzes a comparison between pairs of individual wavelengths and/or the ratio of the differential of signals at each wavelength. Inclusion of multiple emitters at multiple different wavelengths enables monitoring of multiple analytes.

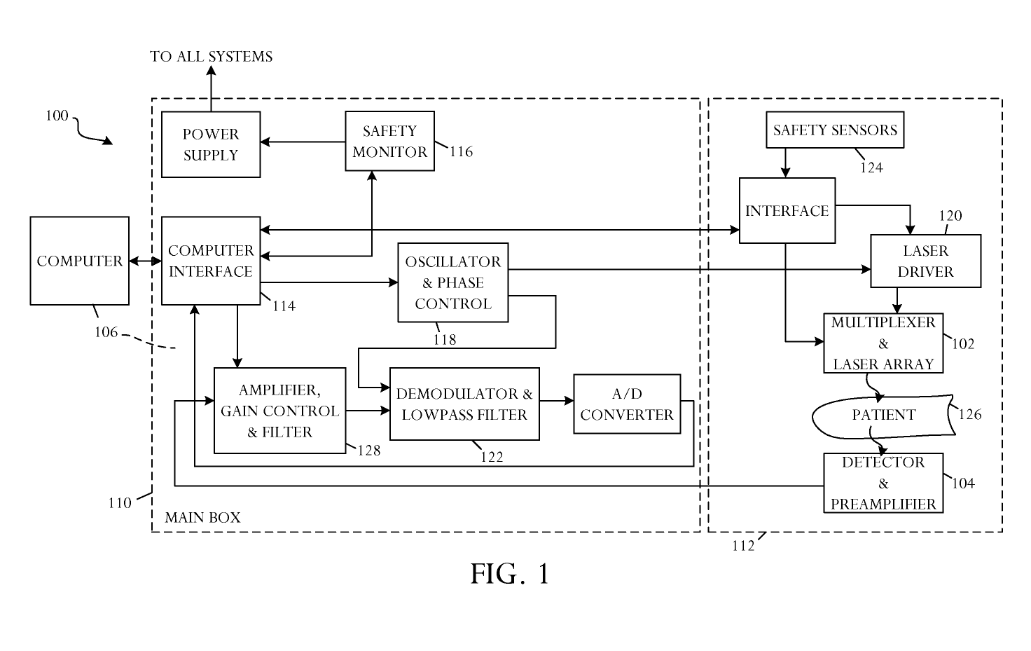

Referring to FIG. 1, a schematic block diagram depicts an embodiment of a tissue sensor 100 configured to monitor multiple physiological parameters. In a particular configuration, the tissue sensor 100 comprises multiple emitters 102 configured to emit narrowband light into biological tissue at a plurality of selected wavelengths including at least one wavelength indicative of a profile for at least one selected analyte and a reference wavelength at which absorbance for the at least one selected analyte is negligible. The tissue sensor 100 further comprises one or more photo detectors 104 configured to detect light at wavelengths that emerge from the biological tissue.

In another embodiment, the illustrative tissue sensor 100 comprises multiple emitters 102 that emit narrowband light into biological tissue at multiple selected wavelengths and at least one photo detector 104 that detects light at wavelengths that emerge from the biological tissue. A logic 106, for example a processor, central processing unit, signal processor, digital signal processor, state machine, logic circuit, or the like, can be configured to sample a signal indicative of the detected light and determine absorbance values evoked by individual emitters from the signal. The logic 106 determines analyte concentration from ratios of the absorbance values for pairs of individual emitters. The logic 106 is defined broadly to comprise a computer or processor external or internal to the main box 110 and may include various circuits and components inside the box 110.

The logic 106 can energize the emitters in a sequence and determine a differential time series for the absorbance values evoked by the individual emitters for usage in determining analyte concentration.

The logic 106 can be configured to control circuits, for example demodulator 122 and amplifier 128, that amplify the signal and remove high-frequency modulation from the amplified signal, thereby forming absorbance values. In some embodiments, the logic 106 can determine a first time-derivative from the amplified signal to form the absorbance values.

In a particular example embodiment, the emitters 102 can comprise multiple Vertical Cavity Surface Emitting Lasers (VCSELs) that emit narrowband light in narrow line widths for a selected spectral range that contains absorption peaks for one or more monitored analytes. In a particular sample embodiment or embodiments, the Vertical Cavity Surface Emitting Lasers (VCSELs) can be selected that emit narrowband light in narrow line widths for emitted light selected from a 600-1400 nm spectral range that contains absorption peaks for multiple analytes. Some embodiments may include one or more VCSELs that emit narrowband light at a reference wavelength that is isobestic for oxyhemoglobin and deoxyhemoglobin. Some embodiments may include one or more VCSELs that emit narrowband light at a wavelength that is indicative of ferritin concentration. Some embodiments may include one or more VCSELs that emit narrowband light at a wavelength that is indicative of glucose concentration. Some embodiments may include one or more VCSELs that emit narrowband light at a wavelength that is indicative of oxyhemoglobin and deoxyhemoglobin concentration. Some embodiments may include one or more VCSELs that emit narrowband light in narrow line widths at wavelengths selected for distribution across a near infrared spectrum and selected to neutralize masking effects of water.

For sensor configurations that include VCSELs selected for monitoring more than one analyte, the logic 106 can be programmed to determine analyte concentration from ratios of the determined absorbance values for multiple pairs of individual emitters. The ratios can be weighted by weighting factors predetermined by back-projection of an error between a target analyte concentration and a predicted value based on previously determined ratios.

In various configurations, the logic 106 can be selected or designed to measure at least one analyte concentration selected from a group comprising glucose, absolute oxygen saturation, total hemoglobin, ferritin, hydration, and others.

The illustrative sensor 100 includes a main box or housing 110 and a probe 112. A computer interface 114 is coupled to the logic 106 and enables the logic 106 to control sensing, measurement, analysis, and display of results for the system.

The main box 110 includes a safety monitor 116 which may have an analog to digital converter (ADC) to enable reading of each supply voltage to insure that the voltage is at safe levels. The safety monitor 116 can include a watchdog circuit to shut the system down if the computer fails to maintain control of the supply voltage.

The illustrative housing 110 also includes an oscillator and phase control 118 that supply a reference signal for a laser driver 120 and a demodulator 122. The oscillator and phase control 118 is configured to adjust demodulator phase to compensate for variable delays in throughout the sensor 100.

The probe 112 includes safety sensors 124 to monitor the temperature of the laser array 102 and the detector 104. The safety sensors 124 include an independent sensor for the laser current, and a separate watchdog to deactivate the laser driver 120 if the computer 106 stops communicating with the probe 112.

The laser driver 120 is programmable and allows an adjustable current, modulated by the signal from the oscillator, to be driven through a particular laser 102 in the array which is selected by the multiplexer.

After passing through patient tissue 126, non-absorbed laser light is detected by the photodiode detector 104, amplified by a preamplifier, and sent to an adjustable amplifier 128 in the main box 110. The amplifier 128 also includes gain control and a filter so that the signal is amplified and filtered to remove noise.

The signal is combined with a reference signal from the oscillator in the demodulator 122, and the demodulated signal is filtered and converted to a digital signal to be returned to the logic 106.

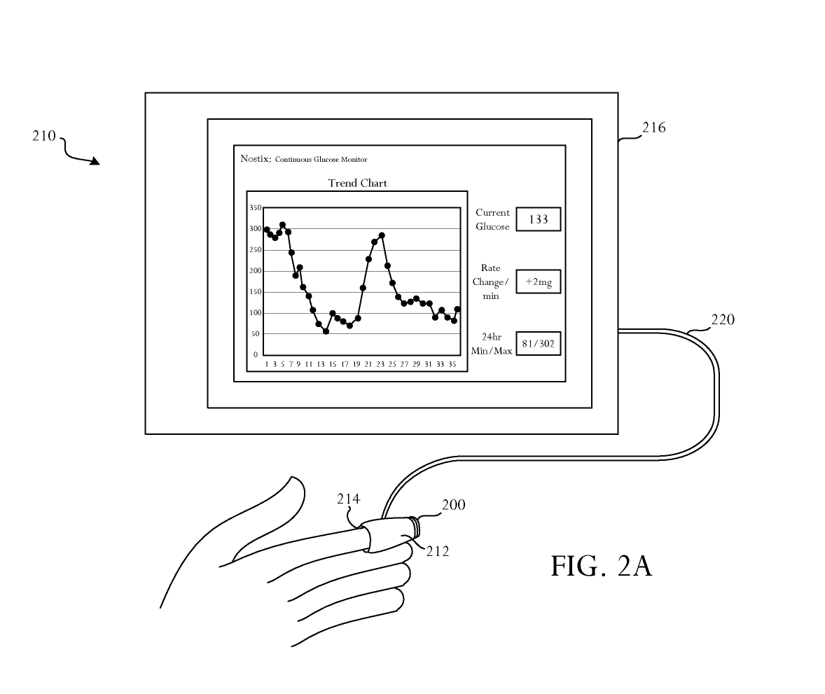

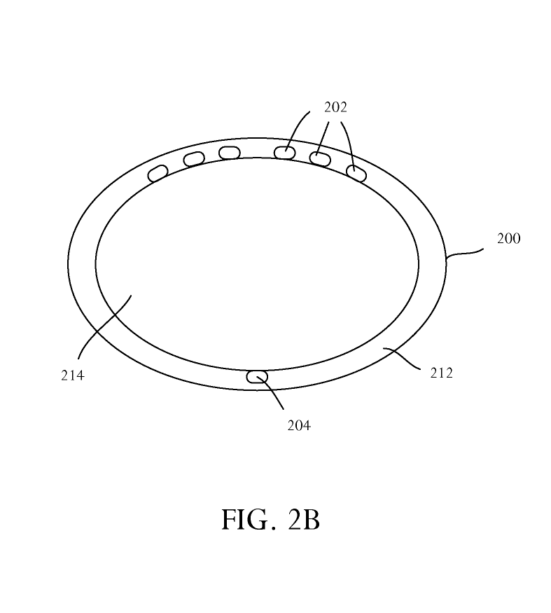

Referring to FIGS. 2A and 2B, an overhead pictorial view and a cross-sectional cut-away view illustrate an embodiment of a tissue sensor 200 in a configuration as part of a critical care monitor 210. The tissue sensor 200 comprises a fingertip sleeve 212 formed about a fingertip cavity 214, multiple emitters 202 arranged on an inner surface on a first side of the fingertip sleeve 212, one or more photo detectors 204 arranged on an inner surface on a second side of the fingertip sleeve 212 across the fingertip cavity 214 from the first side of the cavity. A display 216 coupled to the logic configured to display a parameter indicative of the analyte concentration.

The illustrative critical care monitor (CCM) 210 has two principle components. One component is the sensor 200 designed to be worn on a finger or thumb tip. Several vertical cavity surface emitting lasers (VCSELs) 202 are mounted on one side of the sensor. VCSELs are optical light sources of tiny size, for example approximately the size of a grain of pepper. VCSELs are tuned to specific wavelengths that are directed into the patient's finger. A photodetector is mounted at a side of the sensor 200 opposite the emitters to measure the amount of light at each wavelength that emerges through the finger. Target analytes absorb a portion of the light as the light passes through tissue, and the detector measures a value for the absorption, indicating the amount of a target analyte present in the tissue.

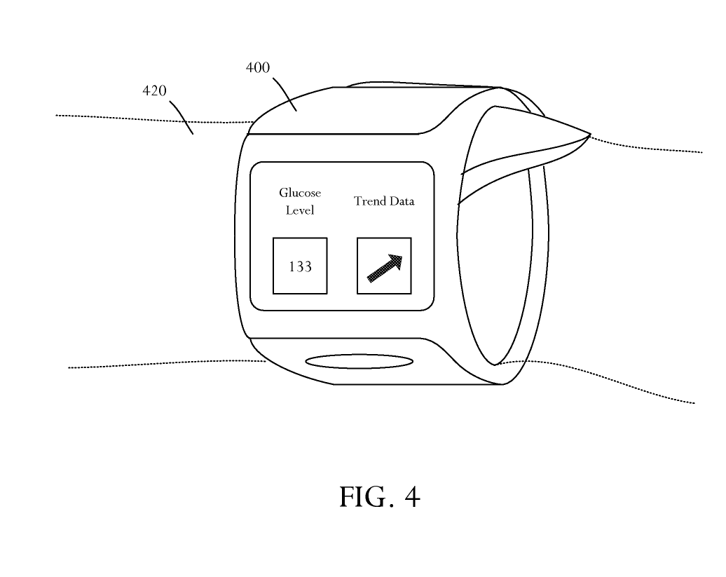

The sensor 200 is connected by a cable 220 to a second component 216, a display/processor, which can range in size from the depicted bedside monitor size to a wearable, battery powered wristband as shown in FIG. 4, depending upon the application. The intensity of the light emitted by a VCSEL is sufficient to produce accurate measurements at only a fraction of the intensity allowed by the United States Food and Drug Administration (FDA) and thus should not present a safety hazard to either the patient or clinician. VCSEL emitters have improved focus, and more powerful, controllable photonics in comparison to conventional emitters such as the LEDs used in pulse oximeters, enabling the CCM 210 to supply measurements with improved accuracy.

In various embodiments, the critical care monitor 210 can include a logic configured to calculate at least one parameter selected from blood volume, core body temperature, and others.

The tissue sensor 200 enables acquisition of many different types of information. The logic implemented in a critical care monitor 210 can be configured to non-invasively and continuously collect one or more data parameters in real-time selected from a single patient status parameter, an absolute value of a single patient status parameter, time trend information for a single patient status parameter, rate of change information for a single patient status parameter, relative comparison information for a plurality of patient status parameters, relative time trend information for a plurality of patient status parameters, and others.

The critical care monitor 210 can be used for long term monitoring. For example, the logic operating in the monitor can be configured to continuously monitor indices of inflammation and injury. In some applications, the logic can be configured to monitor dosage for a therapy.

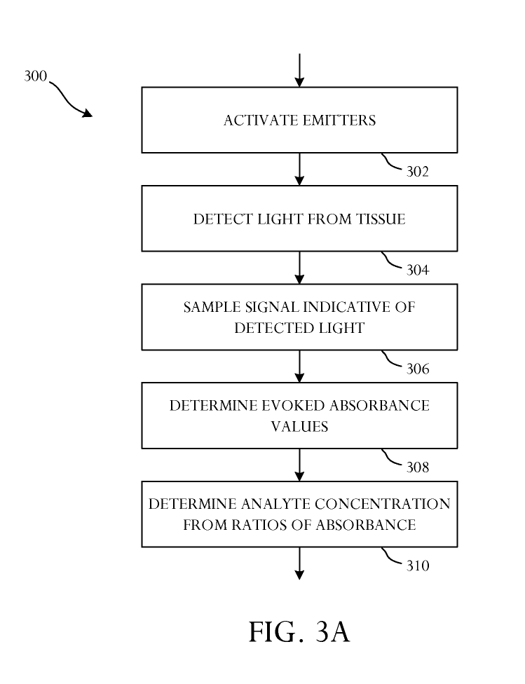

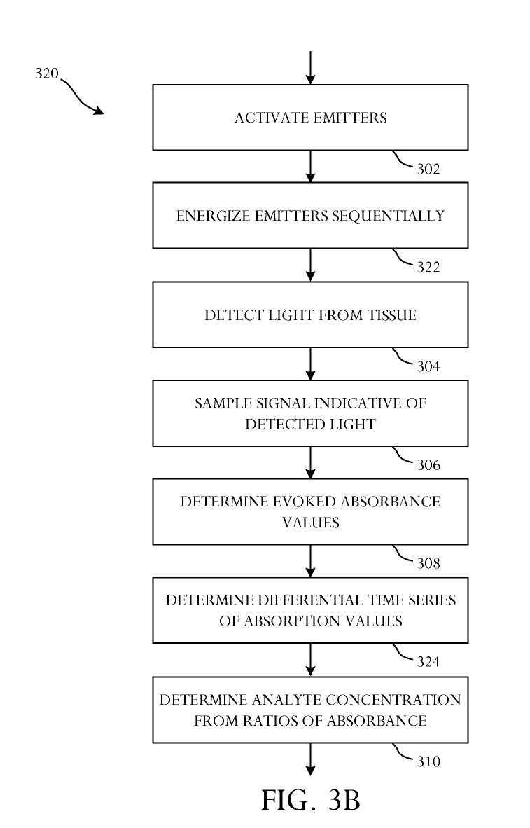

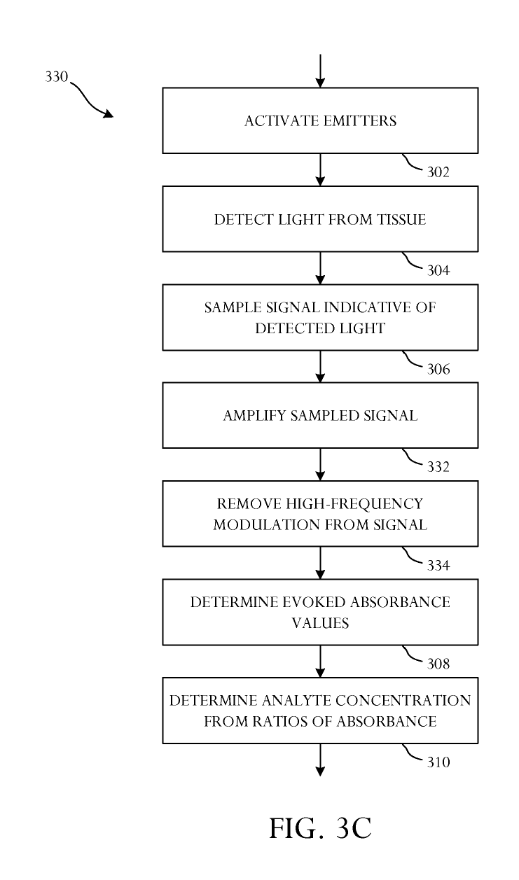

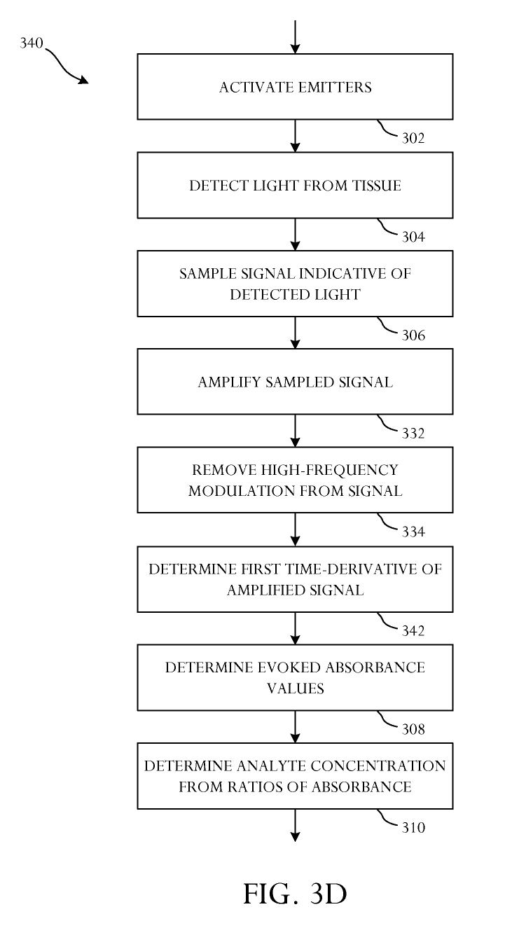

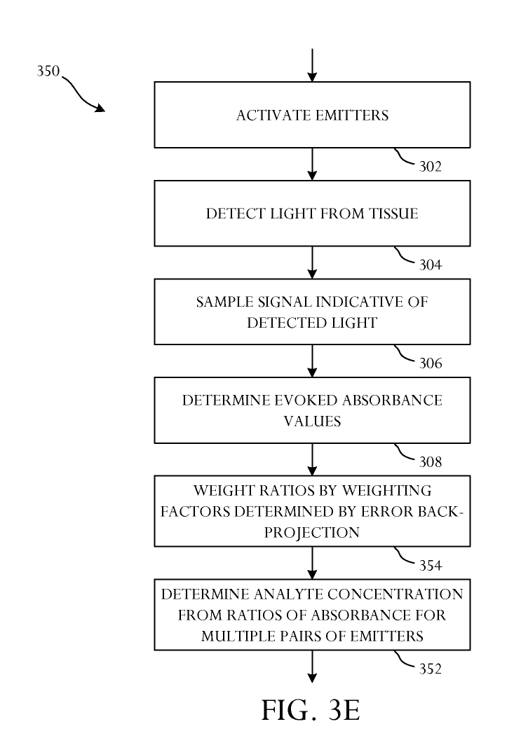

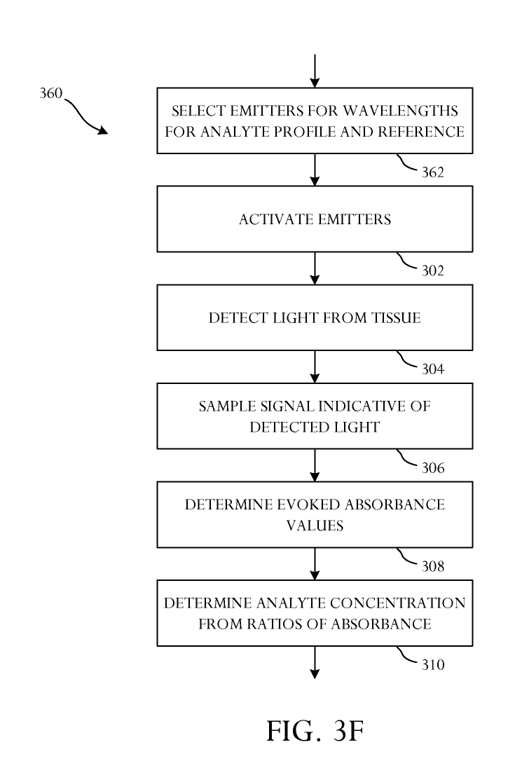

Referring to FIGS. 3A through 3F, multiple flow charts illustrate several embodiments of aspects of embodiments of a method 300 for noninvasively measuring the concentration of at least one analyte. As depicted in FIG. 3A, the method 300 comprises activating 302 multiple emitters to emit narrowband light into biological tissue at multiple selected wavelengths and detecting 304 light at wavelengths emerging from the biological tissue. A signal indicative of the detected light is sampled 306 and analyzed. Absorbance values evoked by the emitters are determined 308 from the signal and analyte concentration is determined 310 from ratios of the absorbance values for pairs of emitters.

Referring to FIG. 3B, in some measurement configurations 320, individual emitters of the emitter group can be energized 322 in a sequence, enabling determination 324 of a differential time series for the absorbance values evoked by the individual emitters for usage in determining 310 the analyte concentration.

As shown 330 in FIG. 3C, the signal indicative of detected light can be amplified 332 and high-frequency modulation removed 334 from the amplified signal to form the absorbance values.

In some arrangements 340, as shown in FIG. 3D, a first time-derivative can be determined 342 from the amplified signal to form the absorbance values.

The illustrative method 300 shown in FIG. 3A depicts a technique for tissue spectra acquisition. In an example operation, a tissue spectral measurement can begin by energizing 302 one VCSEL emitter of a unique wavelength which is positioned on the skin with a high frequency modulated current for 10-15 ms. Emitted light passes through the skin into tissue where much of the light is scattered and a smaller amount of light is absorbed by various analytes. Some of the scattered light emerges from the tissue and energizes 304 a photodetector on the skin. The emerging signal is amplified, the high frequency modulation is removed 308 and the Absorbance stored. Remaining VCSEL emitters can be energized sequentially 322 and the Absorbance stored. The sample rate for six different wavelength VCSELs can be selected to prevent aliasing at the cardiac frequency. A suitable sample rate can be, for example, 50 Hz and tissue spectra measured continuously for about 30 seconds or 1500 spectra. The first derivative of the Absorbance values for each VCSEL has less noise and higher amplitude. A differential time series can be calculated 324 by dividing the difference by the mean of two sequential Absorbance values. For example, a 1498 point differential time series can be averaged to give a single number for each wavelength.

Referring to FIG. 3E, a method 350 can further comprise determining 352 analyte concentration or concentrations from ratios of the determined absorbance values for multiple pairs of individual emitters. The ratios can be weighted 354 by weighting factors predetermined by back-projection of an error between a target analyte concentration and a predicted value based on previously determine ratios.

The weighting factors can be determined using a calibration operation. For example, the ratio of averaged difference to mean averages for every combination of wavelength pairs can be computed. For six unique wavelength VCSEL emitters, fifteen ratios are inputs to an Artificial Neural Network (ANN) and seven intermediate sums are products of the inputs and associated input weighting coefficients. Seven intermediate sums times fifteen ratios result in 105 input weights. The number of output values is determined by a selected number of analyte concentrations up to a maximum of four. Each output value is the sum of the products of the intermediate sums and the associated output weighting coefficients. For seven intermediate sums and four output values, 28 output weighting coefficients are derived. The weighting coefficients are determined by back propagation of the error between a measured actual analyte concentration and the predicted output from the sum of the products of the inputs and weights. The actual analyte concentration can be measured, for example, using an instrument such as a blood analyzer.

Calibration can begin with random weights between 1 and −1. A predicted analyte concentration is computed. Output error is the difference between the predicted analyte concentration and the actual concentration. The output error is initially very large because the weights are random. Very slight changes are made to the weights, resulting in a lower output error. In a feed forward operation, a computation of error and backpropagation of weight modifications is repeated until the output error is lower than a threshold, for example, 5%. Another calibration analyte concentration and a set of input ratios can be loaded into the ANN. The weights are modified until the output error is lower than the error threshold. A minimum of 30 pairs of calibration concentrations and inputs can be used in the calibration. After successful training of a pair a correlation r between the actual and predicted analyte concentration is computed. Training is complete if the correlation coefficient is greater than the correlation threshold, for example r>0.80.

As shown 360 in FIG. 3F, emitters can be selected 362 to emit narrowband light at a plurality of wavelengths that include at least one wavelength indicative of a profile for at least one selected analyte and a reference wavelength at which absorbance for the at least one selected analyte is negligible.

In various example implementations, VCSEL wavelengths can be selected for measurement and/or monitoring of selected analytes. For example, a reference VCSEL wavelength can be selected. In a specific implementation, an 800 nm VCSEL produces a reference wavelength for all analytes of interest because all analytes except oxygen and hemoglobin have no absorbance at the 800 nm wavelength. The 800 nm wavelength is isobestic or has equal molar absorbance wavelength for oxyhemoglobin and deoxyhemoglobin, which do not cause interference at the 800 nm wavelength.

A sensor can be constructed for acquiring a ferritin measurement. Ferritin has an absorption peak in the 825-875 nm range. A VCSEL emitter with wavelength 850 nm can be used to measure ferritin concentration.

A sensor can be constructed to enable oxygen and hemoglobin measurement. Both oxygen and hemoglobin can be determined by measuring oxyhemoglobin and deoxyhemoglobin with a 760 nm VCSEL. Deoxyhemoglobin has a much higher absorbance at the 760 nm wavelength than oxyhemoglobin, which is fortunate because deoxyhemoglobin comprises only 5-15% of the total hemoglobin in healthy human beings.

A sensor can also be configured to measure water concentration. VCSEL wavelengths for water measurement are the same as for glucose.

A particular sensor can be constructed with multiple different VCSELs which are selected to measure a selected set of analytes.

Emitter wavelengths are selected to enable measurement of particular analytes relevant to a particular diagnostic purpose. In a specific example, a tissue sensor may include emitters with center wavelengths at 800 nm, 980 nm, 1020 nm, 1030 nm, 1050 nm, and 1070 nm to usage as a glucose monitor. Wavelength 980 nm is selected for hemoglobin monitoring. As oxygenation of hemoglobin molecules increases and decreases during the respiratory cycle, absorbance caused by oxygen at 800 nm remains the same so that absorbance is independent of oxygenation at 800 nm. Wavelength 980 nm is selected to distinguish water concentration during glucose monitoring because glucose absorbance is almost zero at 980 nm while water absorbance peaks at 980 nm. Wavelengths of 1020 nm, 1030 nm, 1050 nm, and 1070 nm all cover glucose peak absorbance.

In general, emitters can be selected so that multiple wavelengths are positioned in a selected spectral region of interest for a particular analyte or analytes. At least one emitter is selected at a wavelength that is outside the spectral region for the monitored analyte(s) for usage as a reference.

Referring to FIG. 4, a pictorial view illustrates an embodiment of a possible configuration of a wearable Near Infrared Spectrometry (NIRS) sensor 400. The NIRS sensor 400 can be a VCSEL tissue spectrometer that uses Vertical Cavity Surface Emitting Lasers (VCSEL) which emit narrowband light into a tissue volume. Typically, the sensor 400 can be designed to emit light into an appendage such as a finger, arm, or leg, although the NIRS sensor 400 can be configured to transmit or reflect light through any suitable tissue volume. The illustrative NIRS sensor 400 is shown mounted on a wrist 420 which is depicted in phantom. VCSELs are highly suitable tissue spectrometer light sources partly on the basis of a sufficiently small size, for example 300×300×125 um, to be placed directly on the skin in a body worn enclosure. VCSELs typically consume less than 25 mA when activated and, if activated about 1% of the time during a spectral scan, would enable the sensor 400 to be battery powered. VCSELs are noninvasive and safe due to emitting only a small power, for example on the order of 5 mW for 15 ms with a duty cycle of 1%. VCSELs are low-cost relative to other technologies that measure similar diagnostic parameters at least partly of the basis of a capability for manufacture using semiconductor processes. VCSELs have a narrow single-mode bandwidth, for example 1-2 GHz, for emitted light over a 650-1310 nm spectral range that contains absorption peaks for many analytes including ferritin, glucose, hemoglobin, lipids, oxygen, and water.

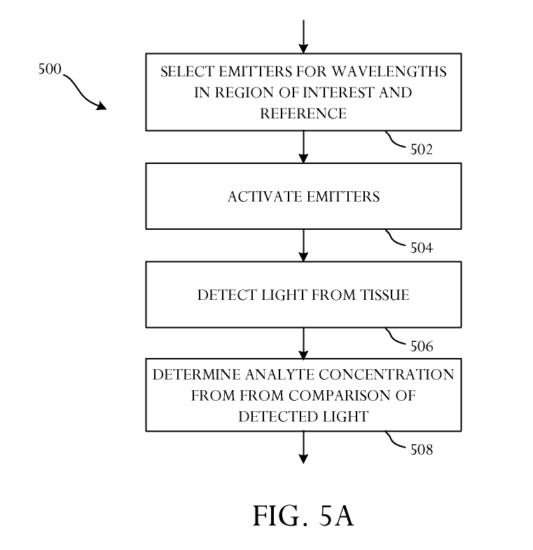

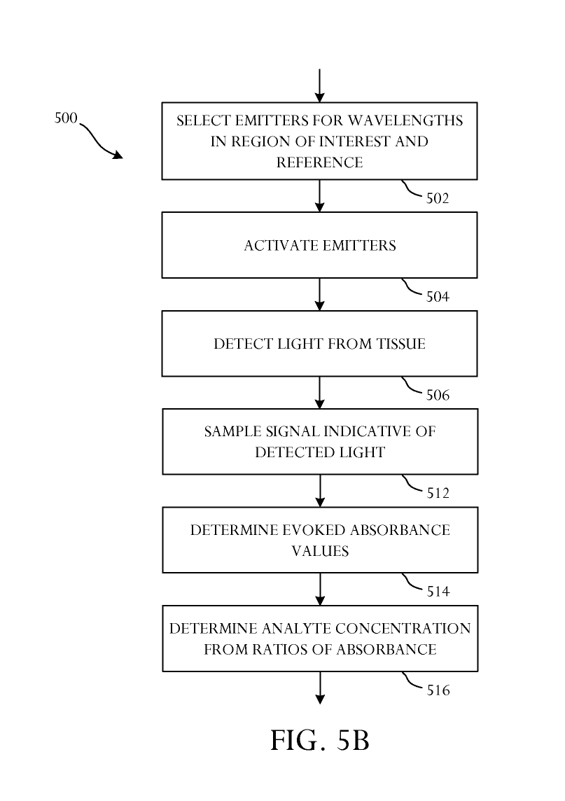

Referring to FIGS. 5A and 5B, schematic flow charts depict embodiments of methods for measuring analyte concentration. As shown in FIG. 5A, the method 500 comprises selecting 502 multiple emitter wavelengths including multiple emitter wavelengths that cover a spectral range for a range of interest for at least one analyte and at least one wavelength outside the range of interest for usage as a reference. For example, a sensor can be constructed for acquiring a glucose measurement. Glucose has an absorption peak in the 970-1100 nm range although water has a stronger absorption in the range. Accordingly, multiple VCSEL emitter wavelengths may be used in the 970-1100 nm range to distinguish the different analytes. Silicon photodetectors lose sensitivity above 1070 nm so a more suitable range may actually be 970-1070 nm. In a particular arrangement, VCSEL wavelengths 980, 1020, 1040, 1060 and 1070 nm can be used for the glucose measurement.

The method 500 further comprises activating 504 the multiple emitters to emit narrowband light into biological tissue at the selected wavelengths and detecting 506 light at wavelengths emerging from the biological tissue. Analyte concentration is determined 508 from a comparison of the detected light for individual emitters of the emitter plurality.

In some embodiments as shown in FIG. 5B, a method 510 can further comprise sampling 512 a signal indicative of the detected light. Analyte concentrations can be determined, for example, by determining 514 absorbance values evoked by individual emitters from the signal, and determining 516 analyte concentration and/or concentrations from ratios of the determined absorbance values for pairs of individual emitters.

The illustrative sensor and sensing techniques can be used for many purposes. For example, the sensor can be used in a Critical Care Monitor (CCM) that can non-invasively and continuously measure a battery of parameters in an ICU setting. In a particular example, the CCM can be used during Surfactant Replacement Therapy (SRT), which can potentially treat patients with Acute Lung Injury (ALI) and the most severe form of ALI known as the Acute Respiratory Distress Syndrome (ARDS). The CCM uses the sensor to enable prediction of at-risk patients who will subsequently develop ALI and perhaps help assess the effectiveness of SRT and/or enable better dosing, delivery and administration strategies. The CCM's predictive capabilities can be exploited to provide earlier treatment with SRT for patients at-risk for developing ALI. The CCM can also be used to enable SRT with more individualized approaches which benefit the patient, a prevention-oriented approach that may be more effective than treating patients who already have established ALI.

Mechanisms involved in the development of ALI and other acute inflammation disorders have been studied for many years. Once ALI has become clinically identified, no treatment is known that can be expected to effectively reverse the damage. Accordingly, focus has shifted from cure of ALI to earlier detection in development of ALI.

No single parameter is known for prediction in the development of ALI. However, conditions characteristic of ALI can be detected using a battery of parameters that monitor different body responses. Accordingly, multiple monitoring capabilities are incorporated into the illustrative CCM. Measurement of the combination of an array of analytes is expected to be more valuable in assessing the status of a critically ill patient than any single parameter. The ability to detect the earliest changes in the parameters can dramatically improve patient outcomes and enable a unique standard of care for patient monitoring. The illustrative CCM enables a broad-based monitoring approach that can improve diagnostic performance over other monitoring standards and acknowledges that multiple monitored factors are interrelated and can be analyzed in combination for an ICU monitor such as the CCM.

NIRS sensing capabilities for the illustrative sensors and associated techniques can be divided into two categories. A first category is direct measurements and includes blood components, or analytes, that can be directly measured by NIRS sensing. A second category is calculated values and represents parameters that can be derived from one or more of the directly measured values. First category, direct measurement values can be attained for glucose, absolute oxygen saturation, total hemoglobin, ferritin, and hydration. Second category, calculated values can be supplied for blood volume and core body temperature.

Early identification and monitoring on a real-time basis for severely ill patients is becoming increasingly desirable. Assessment of severely ill patients' response to treatment by continuously monitoring of readily available surrogate indices of inflammation and injury can assist a clinician by improving diagnosis, treatment and prevention of acute illnesses, such as: sepsis, hemorrhage, shock, Acute Lung Injury (ALI), Acute Respiratory Distress Syndrome (ARDS) and Multiple Organ Failure (MOF), diseases that are generally diagnosed once the patient is in the Intensive Care Unit (ICU). However, the technology can also have value in remote sites such as in battlefields where significant decisions regarding triage can be made more quickly and accurately. Most non-medical people are unaware of the conditions, let alone how the conditions develop or what impact may result.

Various statistical estimates illustrate the desirability of the simple, accurate measurement capabilities of the illustrative sensor and sensing techniques. For example, more than 700,000 cases of severe sepsis, hypo perfusion or shock occur in the US annually, with the worldwide incidence potentially several million cases annually (Sepsis.com/epidemiology). Sepsis has a mortality rate of 28% to 50%, a great and prolonged morbidity, and requires an extensive and costly intensive care treatment (Sepsis.com/overview).

Traditionally, 150,000 cases of ARDS are estimated to occur annually in the US, with a mortality rate of 30-40%.

For every case of ALI or ARDS, depending on the hospital, as many as 10-20 patients may be at risk. Therefore, as many as 3 million patients each year should be monitored annually in the US for development of ALI or ARDS or associated complications.

When the condition of a critically ill patient begins to deteriorate, many health parameters can begin to change rapidly. At the same time, the physician is constrained by a lack of timely, continuous information relating to the unpredictably changing parameters. By way of illustration, results of an arterial blood gas draw can take 30 to 60 minutes to be returned, a time within which a patient's respiratory status may change greatly. To receive the results of a blood test for ferritin, hemoglobin or another parameter may take even longer, and each of the readings are returned as single snapshots of the patient's prior status. Without timely information on trends, decision-making is difficult, so even more tests are typically performed to obtain current information. In many cases, the only alternative available for a physician is to obtain several data points on the same parameter so that some assessment of trends and rates of change can be made. Intermittent, invasive blood tests are slow, expensive, risky and inaccurate—as well as painful to the patient.

The illustrative tissue sensor and Critical Care Monitor (CCM) embodiments can be used in various configurations and arrangements to attain many benefits.

The sensor enables a capability to non-invasively and continuously collect data on a real-time basis. The system can measure an absolute value on a single patient status parameter and track trending and rate of change information on the single parameter. The system can then also monitor and correlate multiple single parameters moving relative to one another to generate a new level of information for diagnosis and treatment.

The illustrative tissue sensors and operating methods enable assessment of a patient's response to treatment by continuously monitoring indices of inflammation and injury. A NIRS sensor can identify how treated individuals respond to a given therapy, and when a therapy could be stopped. Promptly stopping therapies that are not effective or necessary can potentially reduce associated toxicities and therapy expense.

The disclosed tissue sensors, methods, and critical care monitor can be used as a dosage monitor for a therapy such as a drug therapy.

The NIRS-based tissue sensor can be incorporated into many existing products to create new avenues for treatment. The compact tissue sensor can be positioned on catheter tips, in combination ventilators, inside blood pressure cuffs, and the like.

The depicted tissue sensors and operating methods can eliminate human contact with blood samples and eliminate the risk of accidental needle sticks. Similarly, the tissue sensor and CCM can enable cost savings in labor, procedures, risk related to insurance coverage, and prolonged hospital stays, and can reduce patient pain and stress.

The illustrative tissue sensor and diagnostic method can be used for acquiring accurate glucose measurements for patients with ALI. Clinical studies indicate that critically ill subjects including both diabetics and non-diabetics develop hyperglycemia and that intensive insulin therapy increases survival of critically-ill individuals (New England Journal of Medicine 345:1359-1367, 2001). Aggressive insulin therapy reduced hospital mortality by 34%, acute renal failure by 41%, bacteremia by 46%, RBC transfusions by 50%, and polyneuropathy by 44%. Along with correcting the potential pro-inflammatory and other toxicities of hyperglycemia, the protective effect of insulin may be related independently to anti-inflammatory effects perhaps via a resistin type mechanism. Accordingly, measurements of glucose can be another predictor of ALI development in at-risk subjects and earlier administration of SRT reduces not only lung injury but also related secondary, detrimental increases in lung inflammation and hyperglycemia.

The various functions, processes, methods, and operations performed or executed by the system can be implemented as programs that are executable on various types of processors, controllers, central processing units, microprocessors, digital signal processors, state machines, programmable logic arrays, and the like. The programs can be stored on any computer-readable medium for use by or in connection with any computer-related system or method. A computer-readable medium is an electronic, magnetic, optical, or other physical device or means that can contain or store a computer program for use by or in connection with a computer-related system, method, process, or procedure. Programs can be embodied in a computer-readable medium for use by or in connection with an instruction execution system, device, component, element, or apparatus, such as a system based on a computer or processor, or other system that can fetch instructions from an instruction memory or storage of any appropriate type. A computer-readable medium can be any structure, device, component, product, or other means that can store, communicate, propagate, or transport the program for use by or in connection with the instruction execution system, apparatus, or device.

The illustrative block diagrams and flow charts depict process steps or blocks that may represent modules, segments, or portions of code that include one or more executable instructions for implementing specific logical functions or steps in the process. Although the particular examples illustrate specific process steps or acts, many alternative implementations are possible and commonly made by simple design choice. Acts and steps may be executed in different order from the specific description herein, based on considerations of function, purpose, conformance to standard, legacy structure, and the like.

While the present disclosure describes various embodiments, these embodiments are to be understood as illustrative and do not limit the claim scope. Many variations, modifications, additions and improvements of the described embodiments are possible. For example, those having ordinary skill in the art will readily implement the steps necessary to provide the structures and methods disclosed herein, and will understand that the process parameters, materials, and dimensions are given by way of example only. The parameters, materials, and dimensions can be varied to achieve the desired structure as well as modifications, which are within the scope of the claims. Variations and modifications of the embodiments disclosed herein may also be made while remaining within the scope of the following claims. For example, the illustrative embodiments show particular arrangements of emitters and detectors. Many other arrangements are possible including single or multiple emitters and/or sensors. The number of emitters may be one or more, and may be larger than two. The system may have a single detector or multiple detectors. The number of detectors and emitters may be the same or different. Furthermore, although a particular center wavelength is disclosed for the illustrative embodiments, for various applications other wavelengths may be used. Also, the illustrative sensors are shown as devices for mounting on a person's wrist. In other embodiments, the sensors may be arranged for attachment on any other locations on the body.

What is claimed is:

1. A tissue sensor comprising:
a plurality of emitters configured to emit narrowband light into biological tissue at a plurality of selected wavelengths;
at least one photo detector configured to detect light at wavelengths that emerge from the biological tissue; and
a logic configured to sample a signal indicative of the detected light, determine absorbance values evoked by individual emitters from the signal, and determine analyte concentration from ratios of the determined absorbance values for pairs of individual emitters.

2. The sensor according to claim 1 further comprising:
the logic configured to energize the emitter plurality in a sequence and determine a differential time series for the absorbance values evoked by the individual emitters for usage in determining the analyte concentration.

3. The sensor according to claim 1 further comprising:
the logic configured to amplify the signal and remove high-frequency modulation from the amplified signal to form the absorbance values.

4. The sensor according to claim 1 further comprising:
the logic configured to amplify the signal and remove high-frequency modulation and determine a first time derivative from the amplified signal to form the absorbance values.

5. The sensor according to claim 1 further comprising:
the plurality of emitters comprising a plurality of Vertical Cavity Surface Emitting Lasers (VCSELs) that emit narrowband light in narrow line widths for emitted light for a selected spectral range that contains absorption peaks for at least one analyte.

6. The sensor according to claim 1 further comprising:
the plurality of emitters comprising a plurality of Vertical Cavity Surface Emitting Lasers (VCSELs) that emit narrowband light in narrow line widths for emitted light selected from a 600-1400 nm spectral range that contains absorption peaks for a plurality of analytes.

7. The sensor according to claim 1 further comprising:
the logic configured to determine analyte concentration from ratios of the determined absorbance values for multiple pairs of individual emitters weighted by weighting factors predetermined by back-projection of an error between a target analyte concentration and a predicted value based on previously determined ratios.

8. The sensor according to claim 1 further comprising:
the plurality of emitters comprising at least one Vertical Cavity Surface Emitting Laser (VCSEL) that emits narrowband light at a reference wavelength that is isobestic for oxyhemoglobin and deoxyhemoglobin.

9. The sensor according to claim 1 further comprising:
the plurality of emitters comprising at least one Vertical Cavity Surface Emitting Laser (VCSEL) that emits narrowband light at a wavelength that is indicative of ferritin concentration.

10. The sensor according to claim 1 further comprising:
the plurality of emitters comprising at least one Vertical Cavity Surface Emitting Laser (VCSEL) that emits narrowband light at a wavelength that is indicative of glucose concentration.

11. The sensor according to claim 1 further comprising:
the plurality of emitters comprising at least one Vertical Cavity Surface Emitting Laser (VCSEL) that emits narrowband light at a wavelength that is indicative of oxyhemoglobin and deoxyhemoglobin concentration.

12. The sensor according to claim 1 further comprising:
the plurality of emitters comprising a plurality of Vertical Cavity Surface Emitting Lasers (VCSELs) that emit narrowband light in narrow line widths at wavelengths selected for distribution across a near infrared spectrum and selected to neutralize masking effects of water.

13. The sensor according to claim 1 further comprising:
a fingertip sleeve formed about a fingertip cavity;
the plurality of emitters arranged on an inner surface on a first side of the fingertip sleeve;
the at least one photo detector arranged on an inner surface on a second side of the fingertip sleeve across the fingertip cavity from the first side; and
a display coupled to the logic configured to display a parameter indicative of the analyte concentration.

14. The sensor according to claim 1 further comprising:
the logic configured to measure at least one analyte concentration selected from a group consisting of glucose, absolute oxygen saturation, total hemoglobin, ferritin, and hydration.

15. The sensor according to claim 1 further comprising:
the logic configured to calculate at least one parameter selected from a group consisting of blood volume and core body temperature.

16. The sensor according to claim 1 further comprising:
the logic configured to non-invasively and continuously collect at least one data parameter in real-time selected from a group consisting of a single patient status parameter, an absolute value of a single patient status parameter, time trend information for a single patient status parameter, rate of change information for a single patient status parameter, relative comparison information for a plurality of patient status parameters, and relative time trend information for a plurality of patient status parameters.

17. The sensor according to claim 1 further comprising:
the logic configured to continuously monitor indices of inflammation and injury.

18. The sensor according to claim 1 further comprising:
the logic configured to monitor dosage for a therapy.

19. A tissue sensor comprising:
a plurality of emitters configured to emit narrowband light into biological tissue at a plurality of selected wavelengths including at least one wavelength indicative of a profile for at least one selected analyte and a reference wavelength at which absorbance for the at least one selected analyte is negligible; and
at least one photo detector configured to detect light at wavelengths that emerge from the biological tissue.

20. The sensor according to claim 19 further comprising:
the plurality of emitters comprising a plurality of Vertical Cavity Surface Emitting Lasers (VCSELs) that emit narrowband light in narrow line widths for emitted light for a selected spectral range that contains absorption peaks for the at least one selected analyte and the reference wavelength.

21. The sensor according to claim 19 further comprising:
the plurality of emitters comprising a plurality of Vertical Cavity Surface Emitting Lasers (VCSELs) that emit narrowband light in narrow line widths for emitted light selected from a 600-1400 nm spectral range that contains absorption peaks for the at least one selected analyte and the reference wavelength.

22. The sensor according to claim 19 further comprising:
the plurality of emitters comprising at least one Vertical Cavity Surface Emitting Laser (VCSEL) that emits narrowband light at a reference wavelength that is isobestic for oxyhemoglobin and deoxyhemoglobin.

23. The sensor according to claim 19 further comprising:
the plurality of emitters comprising at least one Vertical Cavity Surface Emitting Laser (VCSEL) that emits narrowband light at a wavelength that is indicative of ferritin concentration.

24. The sensor according to claim 19 further comprising:
the plurality of emitters comprising at least one Vertical Cavity Surface Emitting Laser (VCSEL) that emits narrowband light at a wavelength that is indicative of glucose concentration.

25. The sensor according to claim 19 further comprising:
the plurality of emitters comprising at least one Vertical Cavity Surface Emitting Laser (VCSEL) that emits narrowband light at a wavelength that is indicative of oxyhemoglobin and deoxyhemoglobin concentration.

26. The sensor according to claim 19 further comprising:
the plurality of emitters comprising a plurality of Vertical Cavity Surface Emitting Lasers (VCSELs) that emit narrowband light in narrow line widths at wavelengths selected for distribution across a near infrared spectrum and selected to neutralize masking effects of water.

27. The sensor according to claim 19 further comprising:
a logic configured to sample a signal indicative of the detected light, determine absorbance values evoked by individual emitters from the signal, and determine analyte concentration from ratios of the determined absorbance values for pairs of individual emitters.

28. The sensor according to claim 19 further comprising:
the logic configured to determine analyte concentration from ratios of the determined absorbance values for multiple pairs of individual emitters weighted by weighting factors predetermined by back-projection of an error between a target analyte concentration and a predicted value based on previously determine ratios.

29. A method for noninvasively measuring concentration of at least one analyte comprising:
activating a plurality of emitters to emit narrowband light into biological tissue at a plurality of selected wavelengths;
detecting light at wavelengths emerging from the biological tissue;
sampling a signal indicative of the detected light;
determining absorbance values evoked by individual emitters from the signal; and
determining analyte concentration from ratios of the determined absorbance values for pairs of individual emitters.

30. The method according to claim 29 further comprising:
energizing the emitter plurality in a sequence; and
determining a differential time series for the absorbance values evoked by the individual emitters for usage in determining the analyte concentration.

31. The method according to claim 29 further comprising:
amplifying the signal; and
removing high-frequency modulation from the amplified signal to form the absorbance values.

32. The method according to claim 29 further comprising:
amplifying the signal;
removing high-frequency modulation; and
determining a first time derivative from the amplified signal to form the absorbance values.

33. The method according to claim 29 further comprising:
determining analyte concentration from ratios of the determined absorbance values for multiple pairs of individual emitters;
weighting the ratios by weighting factors predetermined by back-projection of an error between a target analyte concentration and a predicted value based on previously determine ratios.

34. The method according to claim 29 further comprising:
selecting the plurality of emitters to emit narrowband light at a plurality of wavelengths including at least one wavelength indicative of a profile for at least one selected analyte and a reference wavelength at which absorbance for the at least one selected analyte is negligible.

35. A method for measuring analyte concentration comprising:
selecting a plurality of emitter wavelengths including multiple emitter wavelengths that cover a spectral range for a range of interest for at least one analyte and at least one wavelength outside the range of interest for usage as a reference;
activating the plurality of emitters to emit narrowband light into biological tissue at the selected plurality of wavelengths;
detecting light at wavelengths emerging from the biological tissue; and
determining analyte concentration from a comparison of the detected light for individual emitters of the emitter plurality.

36. The method according to claim 35 further comprising:
sampling a signal indicative of the detected light;
determining absorbance values evoked by individual emitters from the signal; and
determining analyte concentration from ratios of the determined absorbance values for pairs of individual emitters.

37. An article of manufacture comprising:
a controller usable medium having a computable readable program code embodied therein for noninvasively measuring concentration of at least one analyte, the computable readable program code further comprising:
a computable readable program code capable of causing the controller to activate a plurality of emitters to emit narrowband light into biological tissue at a plurality of selected wavelengths;
a computable readable program code capable of causing the controller to detect light at wavelengths emerging from the biological tissue;
a computable readable program code capable of causing the controller to sample a signal indicative of the detected light;
a computable readable program code capable of causing the controller to determine absorbance values evoked by individual emitters from the signal; and
a computable readable program code capable of causing the controller to determine analyte concentration from ratios of the determined absorbance values for pairs of individual emitters.

38. An article of manufacture comprising:
a controller usable medium having a computable readable program code embodied therein for noninvasively measuring concentration of at least one analyte, the computable readable program code further comprising:
a computable readable program code capable of causing the controller to select a plurality of emitter wavelengths including multiple emitter wavelengths that cover a spectral range for a range of interest for at least one analyte and at least one wavelength outside the range of interest for usage as a reference;
a computable readable program code capable of causing the controller to activate the plurality of emitters to emit narrowband light into biological tissue at the selected plurality of wavelengths;
a computable readable program code capable of causing the controller to detect light at wavelengths emerging from the biological tissue; and
a computable readable program code capable of causing the controller to determine analyte concentration from a comparison of the detected light for individual emitters of the emitter plurality.
